(19) United States
(12) Patent Application Publication
(10) Pub. No.: US 2003/0040870 A1
(43) Pub. Date: Feb. 27, 2003

(54) Automated system and process for custom-designed biological array design and analysis

(76) Inventors: Brooke Anderson, Redmond, WA (US); Siavash Ghazvini, Seattle, WA (US); Patrick Quarles, Redmond, WA (US)

Correspondence Address: Jeffrey B. Oster, CombiMatrix Corporation, 6500 Harbour Heights Parkway, Mukilteo, WA, 98275, US

(21) Appl. No.: 09/838,555

(22) Filed: Apr. 18, 2001

Related U.S. Application Data
(60) Provisional application No. 60/252,880, filed on Nov. 22, 2000.
(60) Provisional application No. 60/198,045, filed on Apr. 18, 2000.

(51) Int. Cl. G06F 17/60; C12Q 1/68; G01N 33/53; G06F 19/00; G01N 33/48; G01N 33/50; G01N 33/543

(52) U.S. Cl. 702/20; 705/2; 435/6; 435/7.1; 436/518

(57) ABSTRACT

There is disclosed an automated system and process for providing a fully automated process for the design, manufacture and analysis of data for biological array (“biochip”) devices. Specifically, there is further disclosed a process and system for obtaining customer orders for custom-designed biochips comprising obtaining desired target sequences from the customer, wherein the target sequences consist essentially of oligonucleotide sequences, polypeptide sequences, or antigens to be bound; creating a sequence content motif for an array, wherein the sequence content motif consists essentially of oligonucleotide sequences, polypeptide sequences, or binding agents designed for complimentary binding; and applying the content motif to a surface suitable for later detection.

### CROSS-REFERENCE TO RELATED APPLICATION

This patent application claims priority from U.S. provisional patent application No. 60/252,880 filed Nov. 22, 2000 that claims priority from U.S. provisional patent application No. 60/198,045 filed on Apr. 18, 2000.

### TECHNICAL FIELD OF THE INVENTION

The present invention provides an automated system and process for providing a fully automated process for the design, manufacture and analysis of data for biological array (“biochip”) devices. Specifically, the present invention provides a process and system for obtaining customer orders for custom-designed biochips comprising obtaining desired target sequences from the customer, wherein the target sequences consist essentially of oligonucleotide sequences, polypeptide sequences, or antigens to be bound; creating a sequence content motif for an array, wherein the sequence content motif consists essentially of oligonucleotide sequences, polypeptide sequences, or binding agents designed for complimentary binding; and applying the content motif to a surface suitable for later detection.

### BACKGROUND OF THE INVENTION

Advances in parallel processing of chemical reactions among biological molecules (e.g., oligonucleotide hybridization, protein-protein binding and interactions, and antigen-antibody binding) are facilitating research activities and automating data gathering and analysis to improve research (particularly medical research) efficiency. While vast amounts of genomic data are becoming available for use in the development of therapeutics and diagnostic tests, the pharmaceutical and biotechnology industries are faced with increasing costs and substantial risks of failure in the drug discovery, development and commercialization process. The lead time for commercializing a proprietary drug now averages 15 years, and the direct and indirect costs of commercializing a successful drug average almost $500 million. Less than 1% of all new chemical entities that are developed by pharmaceutical companies result in pharmaceutical products that are approved for patient use. The pharmaceutical and biotechnology industries are attempting to reduce their costs and risks of failure by turning to new technologies that help identify deficiencies in drug candidates as early as possible in the process so that drug discovery and development becomes more efficient and cost-effective. Additionally, they are searching for ways to expedite their analysis of available genomic data so that they can be the first to bring new therapeutics and diagnostic tests to market.

The discovery and development of new drugs for a particular disease typically involves several steps. First, researchers identify a target for therapeutic intervention, such as a protein, molecule or structure which is either directly involved in the disease or lies in a biochemical pathway leading to the disease. The next step is to identify chemical compounds that interact with the target and modulate the target's activity in a manner that might help reverse, inhibit or prevent the disease. The most promising compounds to emerge from this process advance to the next stage, where synthetic derivatives of the compounds are generated and tested to determine a lead compound. The interactions of these lead compounds with the target and their activity in animal and/or cellular models of the disease are then tested to determine which compounds might be developed successfully into new drugs. The “best” new drug candidates then begin clinical trials in humans.

Recent advances have led to the extensive use in genomics in choosing targets for drug development. This process begins with the discovery and identification of the DNA sequences that make up the genes within the genome. The functions of the discovered genes are then determined so that their role in regulating biological processes and disease can be understood. Information on gene function and disease relevance is used to assess the value of a particular gene or its protein product as a target for drug discovery. Once a target is chosen, high throughput chemistry and other drug discovery methods are used to identify chemical compounds that interact with the target and might help reverse, inhibit or prevent the disease. These compounds are then subjected to the traditional drug development process.

According to industry statistics, pharmaceutical and biotechnology companies world wide spent approximately $55 billion on drug research and development during 1999. Of this amount, approximately 26.7% was spent on drug discovery, 13.9% on toxicology, 32.3% on pre-clinical testing and clinical trials and 27.1% on post-marketing evaluations and other matters.

Biological array processors or “biochips” have potential application in almost all phases of drug discovery and development. In the discovery phase, biological array processors greatly facilitate the process of identifying and validating targets and lead compounds. In the development phases, biological array processors significantly enhance the speed and accuracy of the toxicology, pre-clinical and clinical development process. Moreover, they are expected to play a significant role in monitoring the therapeutic effectiveness of drugs after use. Therefore, there is a need in the art not only to make biochips more readily available but to facilitate the design of the array content and facilitate communication of data developed using biochip arrays. The present invention was made to address this need.

Genetic Variation and Function

Genetic variation and function are mostly due to polymorphisms in genomes, although they may also arise from differences in the way genes are expressed in a given cell, as well as the timing and levels of their expression. Although most cells contain an individual's full set of genes, each cell expresses only a small fraction of this set in different quantities and at different times.

The most common form of genetic variation occurs as a result of variation in a single nucleotide in the DNA sequence, commonly referred to as a single nucleotide polymorphism, or SNP. SNPs are believed to be associated with a large number of human diseases although most SNPs are not believed to have any association with any disease. By screening for polymorphisms, researchers seek to correlate variability in the sequence of genes with a specific disease. A typical SNP association study might require, for example, testing for 300,000 possible SNPs in a patient population of 1,000 individuals. Although only a few hundred of these SNPs might be clinically relevant, 300 million genotyping assays, or tests, must be conducted to complete this study.

While in some cases a single SNP will be responsible for medically important effects, it is now believed that the genetic component of most major diseases is associated with many SNPs. As a result, the scientific community has recognized the importance of investigating combinations of many SNPs in an attempt to discover medically valuable information. In order to understand how genetic variation causes disease, researchers must compare both gene sequence polymorphisms, or conduct SNP genotyping, and gene expression patterns, or gene expression profiling, from healthy and diseased individuals. Biochips are a preferred means for SNP analysis and the networked ability to accumulate and analyze large volumes of such data will be required. The present invention was made to address this need created by biochip uses.

Gene Expression Profiling

Gene expression profiling is the process of determining which genes are active in a specific cell or group of cells and is accomplished by measuring mRNA, which is the intermediary between genes and proteins. Studies of this type require monitoring thousands, and sometimes tens of thousands, of mRNAs in large numbers of samples.

Current Technologies

An array is a collection of miniaturized test sites arranged on a surface that permits many tests to be performed simultaneously, or in parallel, and thus achieves higher throughput. There are many ways to produce arrays, including for example mechanical deposition, bead immobilization, inkjet printing, electrochemical in situ synthesis, and photolithography.

There is a need in the art to improve information processing of data from exposed arrays/biochips and to improve communication of data for customization of biochip arrays. The present invention was made to address the foregoing needs.

### SUMMARY OF THE INVENTION

The present invention provides a process for a manufacturer to obtain customer orders for custom-designed biochips in an automated manner, comprising obtaining desired target sequence(s) from the customer, wherein the target sequence(s) consist essentially of oligonucleotide sequences, polypeptide sequences, receptor binding site, or antigens to be bound; creating a sequence content motif for an array, wherein the sequence content motif consists essentially of oligonucleotide sequences, polypeptide sequences, or binding agents designed for complimentary binding (e.g., hybridization, covalent binding, or protein-protein interactions); and applying the sequence content motif to a surface or within a porous matrix of a volume, suitable for later detection according to the sequence content motif, wherein the communication from the customer and the sequence content motif of each custom-designed biochip is retained within a storage device. Preferably, the desired target sequences are obtained from a database of sequences. Most preferably, the database of target sequences is selected from the group consisting of GenBank, TIGR, Incyte database, private databases and combinations thereof.

Preferably, the step of creating a sequence content motif comprises developing binding regions between a target sequence and a designed capture probe sequence according to consistent reaction conditions, wherein the reaction conditions include temperature and pH. Preferably, the detecting step comprises exposing the custom-designed biochip to a sample to form an exposed custom-designed biochip, and either detecting binding with an instrumentation system designed to obtain a result at each site in a custom-designed biochip to obtain custom-designed biochip exposed data, or shipping the exposed custom-designed biochip back to the manufacturer to determine custom-designed biochip exposed data. Most preferably, the custom-designed biochip exposed data is analyzed by computer using a comparison to the sequence content motif for an array.

Preferably, the surface or the volume on which or within which a sequence content motif is applied is a selected from the group consisting of a solid non-porous surface, a silica-based surface, a porous matrix surface (i.e., porous membrane), a porous volume, a polysaccharide-based surface and layer, glass, and combinations thereof. Preferably, the means for applying sequence content onto the surface or within the volume according to the content motif designed is selected from the group consisting of spotting filly-formed oligonucleotides or polypeptides, in situ synthesis of oligonucleotides or polypeptides by spotting, photolithography of oligonucleotides or polypeptides, in situ synthesis of oligonucleotides or polypeptides by photolithography means, electrochemical-based pH changes in situ synthesis of oligonucleotides or polypeptides, photochemical-based pH changes for in situ synthesis of oligonucleotides or polypeptides, and combinations thereof.

The present invention further provides a system for a manufacturer to obtain customer orders for custom-designed biochips comprising a network-based receiving station for a manufacturer to receive desired target sequences from the customer, wherein the target sequences consist essentially of oligonucleotide sequence(s), polypeptide sequence(s), receptor binding site(s), or antigen(s) to be bound on a surface or within a porous matrix of a volume, or both; a software means for creating a sequence content motif for an array, wherein the sequence content motif consists essentially of oligonucleotide sequences, polypeptide sequences, or binding agents designed for complimentary binding; and a manufacturing system for applying the sequence content to a surface or within a volume or both, suitable for later detection according to the sequence content motif. Preferably, the software means designs sequence content motif for binding to target of oligonucleotide sequence(s), polypeptide sequence(s), receptor binding site(s), or antigen(s) according to uniform melting temperatures, pH, environment, stringency conditions, or other conditions for consistent affinity binding of oligonucleotide sequence(s), polypeptide sequence(s), receptor binding site(s), or antigen(s). Preferably, the system further comprises instrumentation for detecting binding of a sample onto the custom-designed biochip to generate exposure data, wherein the instrumentation resides at the customer or the manufacturer, at a third party or at multiple locations. Most preferably, the system further comprises exposed data to the sequence content motif when the exposed data resides at a first computer-based device and the sequence content motif resides at a second computer-based device or the first computer-based device and the second computer-based device is the same. Preferably, the sequence content motif of each custom-designed biochip is retained within a storage device at the manufacturer. Preferably, the desired target sequences are obtained from a database of sequences. Most preferably, the database of target sequences is selected from the group consisting of public databases, private databases, GenBank, TIGR, Incyte database, private databases and combinations thereof.

Preferably, the creation of content according to the sequence content motif comprises developing binding regions between a target sequence and a designed capture probe sequence according to consistent reaction conditions, wherein the reaction conditions include temperature, pH, stringency, ionic strength, hydrophilic or hydrophobic environment, and combinations thereof wherein a software program having melting temperature, stringency and proton (pH) chemistry algorithms is employed. Preferably, the detecting step that exposes the custom-designed biochip to a sample to form an exposed custom-designed biochip, and either detecting binding with an instrumentation system designed to obtain a result at each site in a custom-designed biochip to obtain custom-designed biochip exposed data, or shipping the exposed custom-designed biochip back to the manufacturer to determine custom-designed biochip exposed data. Most preferably, the custom-designed biochip exposed data is analyzed by computer using a comparison to the sequence content motif for an array data as a template.

Preferably, the surface or volume having a porous matrix on which a sequence content motif is applied is a selected from the group consisting of a solid non-porous surface, a silica-based surface, a porous matrix, a polysaccharide-based surface and layer, glass, and combinations thereof. Preferably, the means for applying sequence content onto a surface or within a porous matrix of a volume, or both, according to the motif designed, is selected from the group consisting of spotting oligonucleotides or polypeptides or in situ synthesis of oligonucleotides or polypeptides, photolithography of oligonucleotides or polypeptides or in situ synthesis of oligonucleotides or polypeptides, electrochemical-based pH changes in situ synthesis of oligonucleotides or polypeptides, photochemical-based pH changes for in situ synthesis of oligonucleotides or polypeptides, and combinations thereof.

### BRIEF DESCRIPTION OF THE DRAWINGS

FIG. 1 shows a rough schematic block diagram of the inventive system linking the customer computer-based communication system to the manufacturer-based servers for custom-designed biochip arrays and analysis of those data generated with each custom-designed biochip array.

FIG. 2 shows a flow diagram of the inventive process by which an array is custom-designed to an experimental need expressed by the customer.

FIG. 3a shows an edit panel in such software in which a researcher has loaded the genetic sequence for the ataxia-telangiectasia locus (from GenBank, accession number u82828, over the Internet) and has specified a mutation at position 94,904 (inserting a G at that location). The researcher could also have specified a target by pasting in a particular genetic sequence and then specifying what the mutation is. The software could also be configured to allow reading in sequence data from other public or private databases.

FIG. 3b shows a list of groups of targets and the contents of one particular group of targets that a researcher has developed. This group has a list of seven targets that the researcher has developed. It also shows that the researcher is selecting one of the targets as something he would like to examine in a target solution. In other words, he is adding that target to an “order” that would be a list of the targets he is interested in examining with a particular DNA array.

FIG. 3c shows a list of targets that the researcher has added to his “order,” which represents a list of targets for which he desires a DNA array to be delivered.

FIG. 3d shows the researcher submitting the order over a network for design and manufacture. He has called it “sample ataxia” and has specified that the array will be helping him determine SNP or mutation data for that set of specified targets.

FIG. 3e shows a screenshot of a piece of software that shows received orders and their status. The “sample ataxia” order is run through the rest of the process, which includes design of probes, layout of the probes in a DNA array format, and starting of the DNA-array synthesis process (making the actual array).

FIG. 3f shows a process by having the sample solution tagged with fluorescent markers and to take an image of the array after hybridization. In this case, relative intensities of light over the locations of the probes is an estimate of how much binding of target has occurred and of the presence or absence of particular targets in solution. The image-analysis program can quantify the intensity data and produce spreadsheets for further analysis. This algorithm that does the analysis of the image data knows (and thus be given data on) the locations of the various capture probes. This program could reside, for example, on a server that receives image data or preprocessed image data (such as just intensity statistics for each array location as opposed to a full image) via a network or on the reader unit itself, which would have to receive information about which probe is where (via a network, CD-ROM, or floppy disk, for example).

### DETAILED DESCRIPTION OF THE INVENTION

Communications networks, such as the Internet, are used to bring the benefits of customized DNA array technology to researchers with the advantages of efficiency of economics and ease of design. Researchers are spared the expense of automated biochip array fabrication equipment and have access to software tools and information that facilitate programming and analyzing custom arrays. The following embodiments of the invention illustrate beneficial uses of wide-area networks, such as the Internet, for designing, ordering, and processing data from biochip arrays.

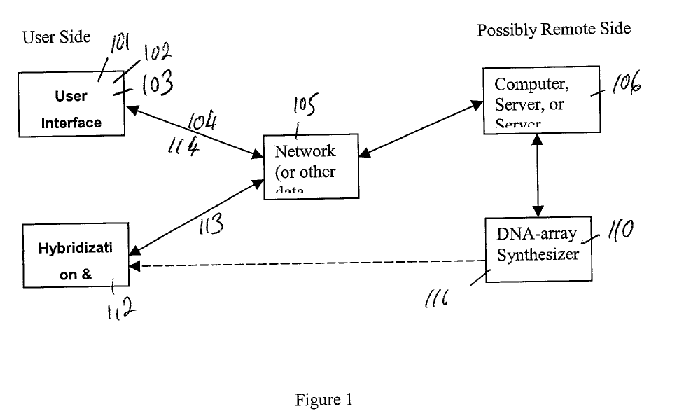

FIG. 1 shows a system whereby a researcher/customer 102 designs a biochip array using a computer 103 at the remote (customer/researcher) location 101. Generally, the array is designed by the customer/researcher (array recipient) by specifying the target sequences or SNP (single nucleotide polymorphism) locations to be tested by the desired arrays. The requested targets 104 or target sequences are sent via a communications network 105 (preferably the Internet) to a local server 106 that is preferably located at or in communication with a server at an array fabrication facility 110. The customer requests (e.g., target sequences, SNPs and the like) are transmitted to another computer 107 that accesses at least one database 108 to complete sequence content motif. Alternatively, the customer's remote computer 103 may access at least one database 108 during the design stage and send a complete sequence content motif to the local server. The local computer sends the sequence content motif to an automated array fabrication unit, which constructs an array 111 according to the sequence content motif. The customer (themselves or through agents or users) exposes the array to test samples. The array is assayed by determining which spots on the array have binding to components of the test samples used. Most preferably, the assay is performed using an assay instrument provided to the customers/researchers/users of the system 112. The assay data 113 are preferably encrypted to prevent tampering and to ensure data security and are then sent to the local server 106 through the communications network 105. A local computer processes the assay data by comparing the result at a particular spot on the biochip array with the sequence content motif (stored as a data template). The processed data are created by the local server (or the customer's computer/server) by comparing the assay data with the sequence content motif stored as a template according to each sequence motif on an array. The local server makes the processed data 114 available for display on the customer's remote computer 103, where the customer can analyze the processed data. Preferably, the assay data 113 is sent to the local server 106 and processed as it is collected. The processed data 114 is preferably immediately available on the local server 106 so that the customer has access to processed data in real time.

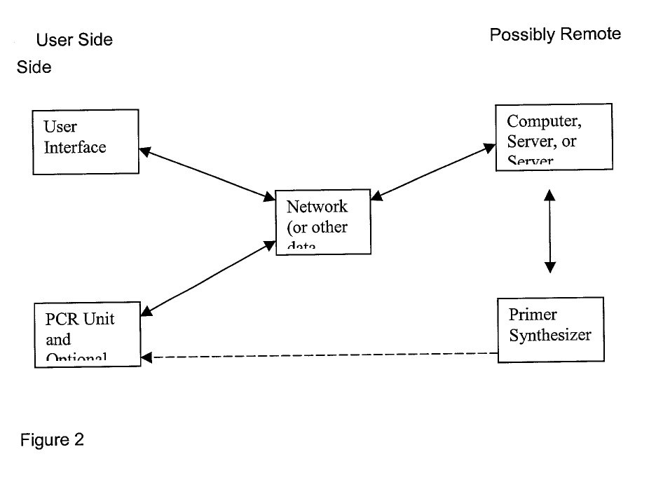

A process by which a customer can use the inventive system for iterative array design is illustrated in FIG. 2. The array design process is simplified by allowing the customer to select target sequences from a database. Once the target sequences have been selected, the target sequences are transmitted to a local server at (or connected to) an array fabrication facility through a network. A local computer connected to the server completes the detailed design specification of the array (sequence content motif) by accessing the database to determine the structure of the probes designed to bind to (e.g., hybridize in the case of oligonucleotides) the target sequences or molecules specified by the customer. A software program located either at the server or at a computer connected to the server calculates appropriate binding probes and the layout of the array, as the sequence-binding motif. In addition the sequence-binding motif is recorded as a template and stored for later analysis when the exposed data are available. The array fabrication and assay process begins when the detailed specification of the array is programmed into an automated array fabrication machine, which constructs the array. The biochip array is exposed to a sample or a plurality of samples containing the targets of interest to the customer to created exposed array assay data. The exposed array assay data are later assayed by comparison to the retained template through network connections or directly if the template is located as the customer facility. The data processing steps begin when the assay data are transmitted to a computer having a templates database, which processes the data and makes the data available to the customer on the local server. During the data analysis process, the customer to decides whether the biochip array content should be modified for optimal use with the customer's sample. If the biochip array content requires modification of the sequence content motif, then the process of sequence motif content design improvement begins. The customer can manually select the sequence modifications, use web-based utilities to select the sequence modifications, or the sequence modifications can be made automatically according to preset capture probe criteria. The improved sequence motif content design is transmitted to a local computer, which translates the customer's modifications into a detailed sequence motif content, by reference to an appropriate database. The biochip array is fabricated as before, but with the modified sequence motif content. The modified biochip array is exposed to the target sample, assayed, and the assayed data is processed as before. If still further modifications are required, the process is repeated. Once the biochip array is optimized, it can be produced in larger quantities for tests of related target samples.

Designing and Specifying DNA Arrays

Custom-fabricated DNA arrays allow researchers/customers to take advantage of the growing databases of DNA sequences available for, for example, analysis and discovery of SNPs (single nucleotide polymorphisms) and for expression of DNA into RNA to cell regulation, pharmacogenomics and toxicity testing. The probes are comprised of stretches of DNA with known sequences that are covalently bound to a substrate. Each site contains many probes and is spaced far enough from adjacent sites to be distinguishable. The inventive process for custom-designing a biochip array allows customers to design biochip arrays by specifying the oligomer sequence that will comprise the probe at each site of a biochip array, by specifying the targets requiring complementary probes by reference to a database identifier, or by specifying targets requiring complementary probes by name and reference to features (e.g., “human BRCA1 unknown at locations 185, 1024, and 13013” or “human BRCA1 unknown from positions 185 to 215”). The inventive method can also help customers design primers for multiplexed PCR (polymerase chain reaction), provide a DNA sequence alignment tool and provide other utilities to help customers design their arrays. The customer's design is sent to the manufacturer server computer over a network (either internal or external). The design is forwarded to a computer that completes the detailed array specification by accessing the referenced sequences from one or a plurality of databases, specifying the full oligomer sequences of the capture probes at each site, and formatting the content specification as required by the automated DNA array fabrication machine.

Fabricating Oligonucleotide Arrays

In a preferred embodiment, oligonucleotide probes are synthesized in situ using an array of electrodes on a semiconductor chip, wherein the oligonucleotides are synthesized on a porous matrix volume located over the electrodes (in situ electrochemical-based manufacturer of DNA microarrays). Overlaying the electrode array is a porous membrane on which the probes are synthesized. The probe sites on the DNA array are matched in two dimensions to the electrode sites on the electrode array. The probes are extended one base at a time by adding the next base specified in a pre-programmed sequence to the 5′ end or the 3′ end of a growing probe. Phosphoramidites nucleotide precursors having a labile blocking group are the nucleotides added to the growing ends of probes. They are preferably modified by addition of dimethoxytrityl (DMT) to the 5′ hydroxyl of the sugar moiety as a preferred blocking group. This modification prevents newly extended probes from further growth by blocking the addition of bases to the 5′ ends of the probes. The oligonucleotide biochip array can selectively remove the DMT protecting groups at particular sites on the biochip array during the fabrication process by the electrochemical generation of acid. Similarly, other oligomers are synthesized by using monomers with acid-labile blocking groups that will be cleaved when the pH in a specified region of a volume in a porous matrix is altered (to a more acid pH). The acid (protons) generated is localized to a particular array site by the acid produced by the electrodes through the current applied to the electrodes. The electrodes are immersed in a buffer or acid scavenger solution and preferably have a porous reaction layer or volume, which helps to hinder diffusion of the electrochemically generated acids. This creates a defined volume (“virtual flask” where the pH is shifted over the electrode and the distinct volume where the next monomer is placed on a growing oligomer.

The customer, researcher or user exposes the custom-designed biochip array to the target sample (containing a probe or marker), either manually or in an automated hybridization apparatus. The hybridization or binding pattern generates an exposed custom-designed biochip where the location of the probe or marker on the target sample delineates sites where binding or hybridization has occurred.

Analysis and Improvement of Biochip Arrays

An aspect of the invention provides a web-based or wide-network-based utility to facilitate the customer's analysis of the processed data from the exposed custom-designed biochip. This utility is customizable so that the customer can indicate the algorithms to be performed for analysis. Pattern recognition and other analysis tools are available from the server via the Internet or other wide-area network used. Once configured to process the array data according to the customer's specification, the utility can interpret array patterns as the array is being assayed. The utility also provides tools to iteratively improve array design. For example, the utility provides statistics based on the results of an array experiment that help the customer design an improved array. The utility suggests specific improvements to the array, such as changes in sequence to particular probes, the elimination of probes that do not interact with the customer's targets, or the addition of probes to test against the customer's targets. A new custom biochip array is fabricated as above, but design changes based on the improvements to the original array are included in the new array. The process is repeated until an array is produced that is optimal for use with the customer's targets. This iterative procedure can be automated, thus requiring little or no input on the part of customers in the optimization of their arrays.

Other embodiments of the present invention can be recognized by those skilled in the art. For example, the design process does not necessarily have to occur at a remote location, but can occur at the array fabrication location. The entire invention is operable at a single location through an intranet or other local area network instead of the Internet. The invention is not limited to providing and analyzing DNA arrays, but can be practiced on any type of array that can be designed, fabricated, and/or analyzed.

An example implementation for studying gene expression is similar to the example for detecting mutations. Again, a researcher develops lists of targets; submits the list of targets for design, layout, and synthesis; hybridizes to the array; and gathers hybridization data. The differences are that the target list is different, representing genes, which can be specified in DNA format, RNA format, or cDNA format; and the probe design and data analysis are different so as to be suitable estimating graded amounts of material present in the sample solution and not just whether or not a particular piece of genetic material is present in solution.

Typically, this probe-design and data-analysis step involves designing probes to selectively capture particular targets in solution. Typically, one specifies conditions that each probe is to satisfy, such as having a melting temperature against its intended capture target within a certain allowed range, having melting temperatures against targets that it is not to capture below a certain value, not having hairpin structures within the probe, possibly having various delta G (change in Gibbs free energy) or change in other thermodynamic values (such as enthalpy, entropy, etc.) against the intended target vs. other targets in solution, etc. The detection process typically involves marking the targets in solution with a fluorescent probe and again estimating amount of material in solution in correlation to the intensity of fluorescence at an array location after hybridization. It can also involve comparing one target solution to another to see how they compare in expression of various genes by comparing intensity data from one array hybridized with one solution to another identically designed array hybridized with another solution. Or, to get around array-to-array variance, one can label one target solution with one fluorescent dye and the other target solution with another fluorescent dye and then hybridize both solutions to the same array and judge the ratio of intensities of the two dyes at each location in the array.

Rather than doing one test on one sequence of DNA at a time, a researcher can do a multitude of tests on various sequences of DNA all at the same time. In the following, “array” will be taken to mean simply a collection of materials that are to be processed, tested, or used in a process all at one time. Thus, an array could be spots of DNA affixed to a substrate where each spot can be a different sequence of DNA, a collection of beads with different DNA sequences on each, a collection of spots of different peptides, a collection of spots of different small molecules that might be drug candidates, a collection of spots of different alloys that might be candidates as a battery electrode material, a collection of primer pairs (not affixed to substrate, but just a collection perhaps in different vials or all mixed together) to be used in PCR to amplify up various segments of DNA all in one batch, a collection of single primers, a collection of different oligonucleotides in solution or suspension, etc. A “site” in the array will be one of the individual spots, beads, spots on the beads, primers, oligonucleotide sequences in solution, etc.—i.e., it represents one of the materials among the many candidate materials in the array.

The prospect of parallel processing gets around the bottleneck of doing one test or processing one candidate material at a time. However, in cases of large arrays that include a large number of individual sites in the array, new bottlenecks can appear such as deciding what to put in the array (i.e., which material to put at each site), building the array (building the collection of materials), reading the results of the resultant use of the array, interpreting the results, etc.

User Interface

The present invention further provides a user interface that a user can employ at a location that might be different from or remote from the site of manufacture of the array. This interface can provide the user with a way to specify the composition of each material at each site or, more preferentially, a way to specify a task or the type of results that are desired from the use of the array or the testing that the array will undergo. For example, a user might specify that he or she is interested in knowing if a DNA sample contains a certain set of genes, so the user would specify which genes the array is to be built to detect without specifying what DNA sequence exactly is to be laid down at each spot of the array. In the case where a user does not specify the composition of the site materials, either a human or, more preferentially, a computer program would take the user's specification (via a network or a storage medium if the computer is remote from the user) and from that decide the sequence make up of the capture probes at each site. The interface is deployed as a custom application that runs on a computer at the user's location, an applet that runs over a network, such as the Internet (such as with Java or Active X), a downloadable application, HTML forms, DHTML pages, XML forms, or any other technology that provides for interaction with the user and communication of data.

In a preferred embodiment, the synthesis of the array is automated. A device (again, possibly at a site remote from the user) can take a specification for the capture probe content to be synthesized at each site in the array and build the array from that specification.

### EXAMPLE 1

This example illustrates a gene expression profiling experiment to determine which genes are active in a sample of tissue or a cell culture. The activity of a gene is determined by the concentration of its transcribed mRNA. The mRNA is isolated from the sample and DNA complements (cDNA) are polymerized using the mRNA as a template. The cDNA is constructed at least in part from fluorescently or radioactively labeled nucleotides. The target sample is comprised of labeled cDNA molecules (usually averaging hundreds of bases) with the same sequences as the coding parts of their grandparent genes. The target sample is tagged with a probe. The microarrays comprise sites containing many identical polynucleotide probes usually averaging more than one hundred bases, but sometimes as short as 25 bases or shorter. The microarray is exposed to the target sample and then assayed. The sequence of a particular cDNA target is determined by the site on the microarray at which the target is bound.

Design of a gene expression capture probe requires knowledge of the sequence of genes to be captured or bound to the microarray in order to specify the sequences of their complementary probe DNA. Customers specify the identity of the genes of interest simply by reference to accession numbers to a database such a GenBank, dbEST, and UniGene. The microarray pattern of capture probes is forwarded, via the Internet, to a user. The user (customer) is provided with a microarray that can detect expression of the genes specified by the customer/user. The data gathered from the expression microarray indicates the active genes from the MRNA sample tested.

### EXAMPLE 2

Expression profiling of mRNA from diseased tissue samples can give information as to whether abnormal expression of a gene is the cause of the disease, and if so, which gene is implicated. A drug development researcher who suspects a number of candidate genes are implicated in a particular disease designs an array using a web-based utility to specify those genes. The design is transmitted to a local server at the array fabrication facility over the Internet. A detailed specification for the array is created by accession of the sequences of the targets specified by the researcher and development of complementary probes to those targets. Arrays are fabricated according to the detailed specification and are then provided to the researcher. The researcher exposes at least one array to cDNA capture probes complementary to the mRNA transcribed in diseased tissue, and exposes at least one other array to cDNA targets complementary to the mRNA transcribed in healthy tissue. Alternatively, a single array can be used if the diseased and healthy cDNA targets are labeled with spectrally distinguishable fluorophores. The array or arrays are assayed, and the assay data is sent via the internet to a local server at the array fabrication facility.

The assay data are processed by a computer, and is made available on a server for analysis by the researcher. The researcher can use a web-based, utility to study the differences between gene expression in diseased and healthy tissue. The researcher can use the information from such an experiment to iteratively refine the array, or to guide further experimentation.

### EXAMPLE 3

Polymorphisms are fairly common characteristics of any genome. Polymorphisms are variations within the genome of a species including nucleotide insertions and deletions and variations in the number of repeats of a repeated sequence. Common polymorphisms are single base variations in the genetic code called single nucleotide polymorphisms (SNPs). Most commonly, there are two naturally occurring polymorphs per SNP, e.g., a guanine (G) is replaced by an adenosine (A), but up to four polymorphs per SNP are possible if cytosine (C) and thymine (T) can replace G. Polymorphism discovery research seeks to map out a genome based on the locations of its SNPs.

There are several different methods for polymorphism discovery using DNA arrays. For example, in one method the sequence of a reference target (usually greater than 100 bases, e.g., a gene or other genome fragment) is generally known to the user due to the availability of gene sequence databases. The reference target sequence is conceptually divided into overlapping segments of, for example, 25 bases. (The number of bases is not a critical factor, but it is usually around 25.) Each 25 base sequence (25-mer) differs from the previous sequence in that the first base of the previous sequence is removed, and the last base of the next sequence is the next base in the reference target. In other words, each segment is a 25-base “window” of the target DNA sequence. These 25-mers form the basis for the capture probes of the microarray. If the target DNA sequence is conceptually divided into N 25-mers, then for each of the original N 25-mers, three additional 25-mers are created for a total of 4N 25-mer sequences. The three additional 25-mers created from each original 25-mer are identical to the original 25-mer except that the 13^{th} base (the one in the middle) of each additional 25-mer is a different nucleoside. For example, if the 13^{th} base in an original 25-mer is G, then the three additional 25-mers have the same bases as the original 25-mer, except that the 13^{th} base is A, C, or T.

The 4N capture probes are arranged in a microarray. The DNA array is exposed to a plurality of labeled targets comprising the same gene or genome fragment, but from different sources. If any particular 25 base sequence within the sample targets contains a single nucleotide polymorphism (SNP) at the 13^{th} position, then targets will hybridize not only to the original 25-mer that is complementary to the reference target's corresponding 25-base sequence, but also to one or more of the other three 25-mers that differ by a nucleoside variation at the 13^{th} position. However, if no target contains a 25 base sequence with a polymorphism at that position, then targets will hybridize only to the 25-mer that is complementary to the corresponding sequence of the reference target. This is because the hybridization reaction is much less favorable if there is an uncomplimentary base in the middle of two sequences to be hybridized.

The array is assayed, and the assay data is processed as follows. Each site on the array determined to have hybridized targets is identified and mapped to the reference target sequence. Targets bound to any site corresponding to one of the additional 25-mers is particularly noted, as is the identity of the 13^{th} base of the additional 25-mer. The reference target sequence is thus reproduced, the SNP positions are identified, and the particular polymorphs are specified by identifying the polymorphic nucleosides.

In the design step, customers specify the regions of a genome in which they are interested in finding polymorphisms by reference to a database, such as through an accession numbers (i.e., Genbank). They then forward this information, via the Internet (or another communications network), to a local server at the array fabrication facility. A local computer accesses the database for the DNA sequences referenced by the customer. The local computer designs the original 25-mers and the additional 25-mers to be used as probes, and then composes the detailed specification of the array. This detailed specification is input into the automated array fabrication instrument, which creates the array.

In the processing step, the array is exposed to a collection of targets comprised of the same genes or genomic regions, but from different sources. The array is assayed and the assay data is processed by a local computer. The processed data is available on a local server for the customer to access over the Internet. A web-based utility allows the customer to analyze the processed data in a meaningful way, perhaps using a graphical representation of the reference target with the locations and identities of SNPs indicated.

### EXAMPLE 4

Some polymorphic variations can result in disease or be markers for disease or even prognostic indicators. The iterative procedure for designing a clinical genetic analysis array begins by correlating polymorphisms discovered as described in Example 3 above with particular genetic diseases. A polymorphism detection array is designed as in Example 3, and the design is transmitted over the network to a local computer at the array fabrication facility, which then programs the array into the automated array fabrication machine, which fabricates the array. Target samples obtained from a population known to have a genetic disease are tested on the array and compared to the results of similar tests of targets obtained from a healthy population. The array data from the healthy and the diseased populations are transmitted over the network to the local computer, which processes the data by determining which polymorphisms the diseased population have in common, but which differ from those of the healthy population. Such polymorphisms may be implicated in the genetic disease being studied.

A web-based utility aids in optimization of arrays for detection of disease-producing polymorphisms by removing probes for non-implicated polymorphisms from the arrays. Algorithms for determining whether a polymorphism is implicated in disease are set by the customer, or the implicated polymorphisms may be automatically selected. The identities of probes that have been found to detect targets that indicate genetic disease are stored, either on the customer's computer or on a local computer. Once the customer has found a number of disease-indicative polymorphisms, the probes to detect these polymorphisms are combined into a single array. This array is produced in bulk to provide tools for simple clinical genetic analyses.

The arrays are used to determine individuals' propensity to particular genetic diseases by providing a simple screening test for those diseases. The arrays are also used to diagnose genetic diseases. The key to the probe identities in a genetic analysis array is beneficially kept secret from the customer/clinician, and the assay data from such an array is beneficially encrypted before being transmitted to the service over the network. The steps ensure the privacy of the individual who is being screened or diagnosed. The results of screening tests or diagnoses can be made available to the clinician, or they can be sent directly to the screened or diagnosed individual or to another party if privacy is a concern.

### EXAMPLE 5

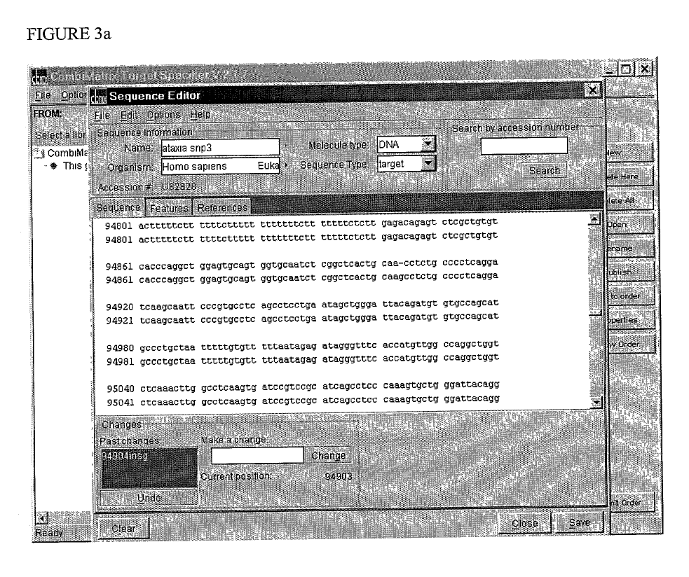

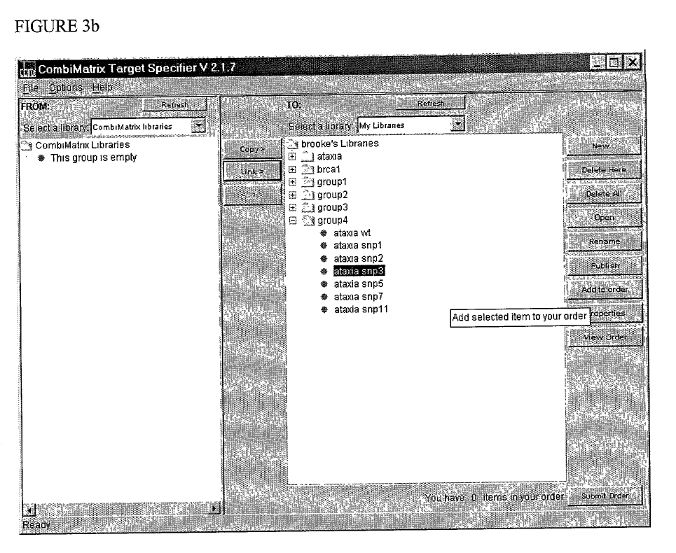

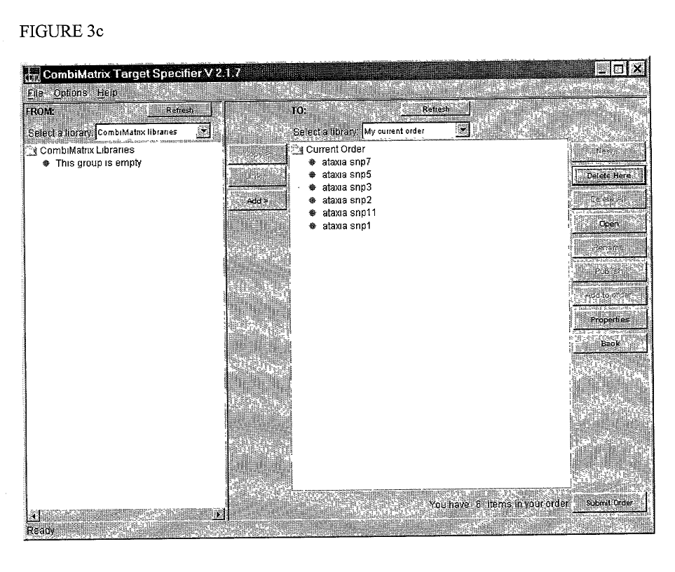

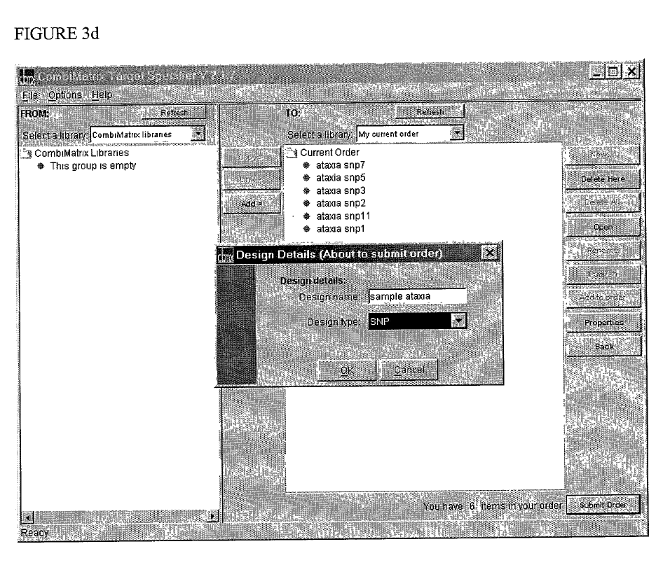

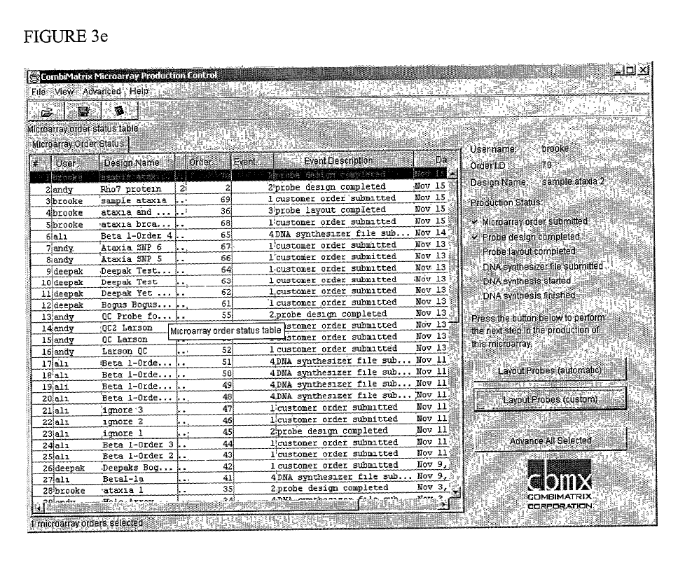

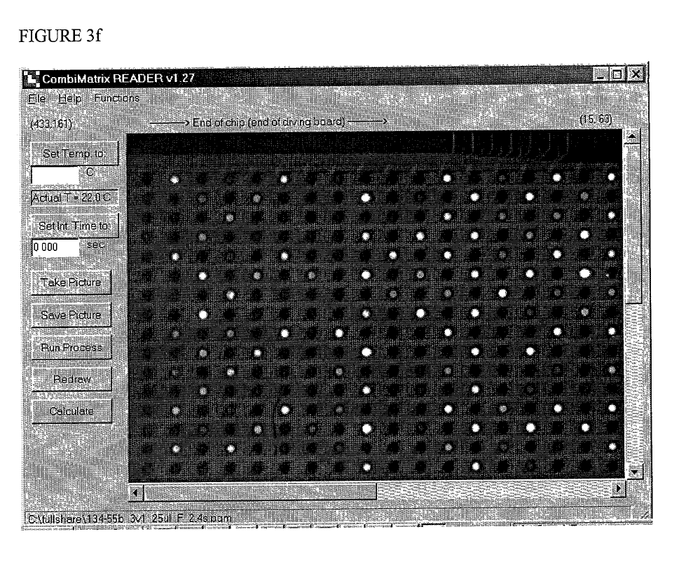

FIG. 3a shows an edit panel in a software program wherein a researcher has loaded the genetic sequence for the ataxia-telangiectasia locus (from GenBank, accession number u82828, over the Internet) and has specified a mutation at position 94,904 (inserting a G at that location). FIG. 3b shows a list of groups of targets and the contents of one particular group of targets that a researcher has developed. This group has a list of seven targets that the researcher has developed. It also shows that the researcher is selecting one of the targets as something he would like to examine in a target solution. In other words, he is adding that target to an “order” that would be a list of the targets he is interested in examining with a particular DNA array. FIG. 3c shows a list of targets that the researcher has added to his “order,” which represents a list of targets for which he desires a DNA array to be delivered. FIG. 3d shows the researcher submitting the order over a network for design and manufacture. He has called it “sample ataxia” and has specified that the array will be helping him determine SNP or mutation data for that set of specified targets.

The list of targets is filed for later reference, and it is ready for probe design software to design probes appropriate to that set of targets and that type of experiment (SNP detection). FIG. 3e shows a screenshot of a piece of software that shows received orders and their status. The “sample ataxia” order can be run through the rest of the process, which includes design of probes, layout of the probes in a DNA array format, and starting of the DNA-array synthesis process (making the actual array).

The probe-design step takes the specified targets and designs a set of probes for each target. Each probe set for each target is designed to allow data analysis such that the likelihood of the target being present in the solution can be estimated. Table 1 (below) gives one possible list of probes that were designed for the “sample ataxia” set of targets (along with some quality-control probes that were designed for the array). In this case, the probes were designed in the following manner. For single-base changes (such as an SNP where an A changes to a C, for example), one probe was made to be the complement of the wild type, overlapping the position of the base change; one probe was made to be the complement of the mutation, overlapping the same position; and one probe was made to be the complement of a different mutation (different from both the wild type and the mutant). For changes that were an insertion or deletion, one probe was made to be the complement of the wild type, overlapping the border of the insertion or deletion; one probe was made to be the complement of the mutation, overlapping the same position; one probe was made to be the complement of a single-base changed version of the wild type, where the single-base change happens for a base just to one side of the position of the mutation; and one probe was made to be the complement of a single-base changed version of the mutation, where the single-base change happens for a base just to one side of the position of the mutation. One can judge if the wild-type probe or mutation probe is more strongly hybridized to than the negative control or controls and also which type (wild type or mutant) is more strongly bound or if they are both approximately equally bound. In this manner, one can develop an estimate of the presence of wild type or mutant and whether the sample is homozygous or heterozygous.

TABLE 1

| CaptureProbe | | Locus AuxInfo | Tm | Start | End |
|---|---|---|---|---|---|
| 1 | tacgccaccagctcc | 194 qc-1 | 55.87 | 1 | 15 |
| 3 | tacacctcctgcacc | 196 qc-3 | 51.98 | 1 | 15 |
| 4 | tggtccgctctcacg | 197 qc-4 | 55.88 | 1 | 15 |
| 5 | ccgataaataacgcg | 198 qc-5 | 46.55 | 1 | 15 |
| 6 | taaatgtcgttcgcg | 199 qc-6 | 48.98 | 1 | 15 |
| 7 | ttggcgaagaaggag | 200 qc-7 | 50.05 | 1 | 15 |
| 8 | gcccggtttatcatc | 201 qc-8 | 48.43 | 1 | 15 |
| 9 | tgattaacgcccagc | 202 qc-9 | 51.05 | 1 | 15 |
| 10 | cttcaggcggtcaac | 203 qc-10 | 51.89 | 1 | 15 |
| 19 | cagttcagtattatcta | 567 12666-wild-a | 42.06 | 29 | 45 |
| | | Wild- | | | |
| | | a | | | |
| 20 | cagttcagcattatcta | 567 12666-snip-g | 46.21 | 29 | 45 |
| | | SNiP- | | | |
| | | g | | | |
| 21 | cagttcagaattatcta | 567 12666-wneg-t | 36.89 | 29 | 45 |
| | | WNeg- | | | |
| | | t | | | |
| 39 | aactgaggtagatggct | 563 65419wild-a | 52.86 | 93 | 109 |
| | | Wild- | | | |
| | | a | | | |
| 40 | aactgaggcagatggct | 563 65419-snip | 56.99 | 93 | 109 |
| | | SNiP- | | | |
| | | g | | | |
| 41 | aactgaggaagatggct | 563 65419wneg-t | 47.69 | 93 | 109 |
| | | WNeg- | | | |
| | | t | | | |
| 59 | tccctaaccagatgaag | 566 86847wild-g | 50.72 | 20 | 36 |
| | | Wild- | | | |
| | | g | | | |
| 60 | tccctaacaagatgaag | 566 86847snip-t | 48.51 | 20 | 36 |
| | | SNiP- | | | |
| | | t | | | |
| 61 | tccctaacgagatgaag | 566 86847-wneg-c | 45.03 | 20 | 36 |
| | | WNeg- | | | |
| | | c | | | |
| 79 | acacattccctggattt | 568 89606-wild-g | 51.61 | 76 | 92 |
| | | Wild- | | | |
| | | g | | | |
| 80 | acacattcactggattt | 568 89606-snip-t | 49.43 | 76 | 92 |
| | | SNiP- | | | |
| | | t | | | |
| 81 | acacattcgctggattt | 568 89606-wneg-c | 47.09 | 76 | 92 |
| | | WNeg- | | | |
| | | c | | | |
| 107 | gggcagaggttgcagtg | 565 94896-wild-a | 59.58 | 93 | 109 |
| | | Wild- | | | |
| | | a | | | |
| 108 | gggcagaggatgcagtg | 565 94896-wneg-t | 53.13 | 93 | 109 |
| | | WNeg- | | | |
| | | t | | | |
| 111 | ggcagaggcttgcagtg | 565 94896-snip-a | 59.98 | 93 | 109 |
| | | SNiP- | | | |
| | | a | | | |
| 112 | ggcagaggcatgcagtg | 565 94896-sneg-t | 54.18 | 93 | 109 |
| | | SNeg- | | | |
| | | t | | | |
| 147 | ttcttctagattttcta | 564 164713-wild-t | 41.18 | 93 | 109 |
| | | Wild-t | | | |
| 148 | ttcttctagtttttcta | 564 164713-wneg-a | 35.99 | 93 | 109 |
| | | WNeg-a | | | |
| 151 | ttatccattattttcta | 564 164713-snip-t | 38.94 | 93 | 109 |
| | | SNiP-t | | | |
| 152 | ttatccatttttttcta | 564 164713-sneg-a | 34.5 | 93 | 109 |
| | | SNeg-a | | | |

The next step is to lay out the probes in an array and to synthesize the array. In this case, software can lay out the probes in a scanned fashion, filling available array spots with these probes (and duplicates of these probes if more array positions are available than are needed for one set of probes), create a file for a DNA-array synthesizer that then (after receiving the data over a network) synthesizes the array, and the array would then be ready for a quality-control check (to validate the synthesis) and then for use by the researcher in his experiment.

At this point, the researcher or a customer can take the array and the sample solution, perform a hybridization and take data from the array. One way of doing this is by having the sample solution tagged with fluorescent markers and to take an image of the array after hybridization, such as the image in FIG. 3f. In this case, relative intensities of light over the locations of the probes is an estimate of how much binding of target has occurred and of the presence or absence of particular targets in solution. The image-analysis program can quantify the intensity data and produce spreadsheets for further analysis. This algorithm that does the analysis of the image data should know (and thus be given data on) the locations of the various probes. This program could reside on a server that receives image data or preprocessed image data (such as just intensity statistics for each array location as opposed to a full image) via a network or on the reader unit itself, which would have to receive information about which probe is where (via a network, CD-ROM, or floppy disk, for example).

### EXAMPLE 6

FIG. 1 lays out one possible configuration of different pieces for the purpose of using oligonucleotide microarrays. In the figure, the various pieces are shown separated, communicating by a network. However, various individual boxes in the figure could be integrated together in any combination. It is shown as the user interface running on a client computer and that the client computer, the hybridization/reader unit, and the server would all be hooked up to the Internet, and that the DNA synthesizer would be hooked into a LAN. However, any piece could be located locally or remotely and hooked up via LAN, Internet, etc.,—just as long as the various pieces can communicate appropriately, getting the information they need from other pieces.

In FIG. 1, the dashed arrow represents delivery of a synthesized array to the user so that it can be put through hybridization. However, the hybridization unit might be combined with the synthesizer so that no physical transference of the array is required.

### EXAMPLE 7

Example 7 describes the operation of the apparatus and methods from a user's point of view. First, the user will specify which targets he or she is interested in getting information about and possibly which are likely to be in the sample (solution). Second, a server or servers (possibly with human intervention or help) will take the specification and design an array for the task. Third, the server will send the array specification to a DNA-array synthesizer that will make the array. Fourth, after an array is made that passes quality-control checks, the array is shipped to the user. Fifth, the user inserts the array into the hybridization/reader unit along with the sample, and the unit does the hybridization, gathering results and sending the results to a server. Sixth, the server processes, interprets, and formats the data and presents it back to the user on a workstation.

Step 1: Target Specification

The user interacts with target-specification software, most preferentially through a Web browser interface or a custom application (working over the Internet). This is shown in FIG. 1 as the “User Interface.” Some tasks researchers use DNA arrays include expression studies and polymorphism studies as described herein. These and other uses of DNA arrays are usually subsets of the general case of putting down segments of DNA in an array such that each segment captures its complementary piece of DNA in solution. Then the user concludes that each site that gets bound to (with material from the sample) equates to that site's complementary DNA being in solution.

The computational task of interpreting a specification by the user can be easier such as in the case where the user specifies the full sequence of any material likely to be in the solution and specifies which from among the sequences specified are the ones to be captured (or bound to) at the sites of the array. Or the task might be more complicated such as in the case where the user simply specifies genes that he wants to identify in the solution, such as something like “human BRCA1 with the mutation 185delAG” as a specification of one target or query (i.e., to decide whether or not that target is in solution or how much of it is in solution in the case of a differential test). Or the user might want to know the sequence of a particular piece of DNA, knowing parts of the sequence, but being unsure of the identity of a base here and there or even of some particular segment, and thus might specify something like “human BRCA1 unknown at locations 185, 1024, and 13013” or “human BRCA1 unknown from positions 185 to 215” and want to know what bases are at the locations specified. Or the user might specify an accession number from Genbank instead the name for the gene or genetic material. The complication can come out of being able to handle many different types of specifications as opposed to a rigid format that is always the same regardless of task.

In some of the above cases, the server side would need to do more processing to develop the DNA sequence of the target being specified, interacting with the a database to pull out the mRNA sequence for BRCA1, using the mutation specification to set the mutation, then using a database again to translate back into DNA.

Step 2: Array Design

Microarrays are tending to higher densities. Automated (or semi-automated) array-design software can implement mathematical models and heuristics to help speed the process of designing particular probes given a list of targets to capture. Array design might also be an iterative process. For example, the user might specify targets or other initial input, view the result of the first pass at array design and possibly some associated statistics or simulated hybridizations and results, and from that decide to change some input parameters, heuristics, or particular probes (to be designed again). This process might be repeated until the user is satisfied with the probe array.

One design process is represented in FIG. 1 as being internal to a server or servers at a (possibly) remote site; or, if there is user interaction at each design iteration, it is represented by the link from the server through the Internet back to the user's computer (which would be running a browser-type interface to a portion of the design software or perhaps custom front-end software that, again, would communicate to the server through the Internet). Or the server could be the user's own computer or a server at the user's site.

Step 3: Array Synthesis

After the array design is complete, the array specification is sent to a synthesizer that then makes the microarray by adding capture probes, also called “content.”.

Step 4: Ship to User

The array is checked for quality. Passed arrays could be sent, via overnight courier, to the user the next day.

Step 5: Hybridization & Reading

The user would put the array and the sample he or she is interested in interrogating into a hybridization unit or a combined hybridizer/reader unit. The hybridization unit carries out the hybridization reaction and images the results. These data could then be sent to a server that could do any required processing and formatting of the data, or it could be done on the hybridization unit's internal processor.

Step 6: Get the Results

After the server processes and formats the hybridization data, it can be sent back to the user or made available for him to view, again possibly using a browser or custom front-end software.

### EXAMPLE 8

FIG. 2 lays out one possible configuration for the purpose of using PCR-primer arrays. In the figure, the various pieces are shown separated, communicating by a network. However, as in the DNA-array example, various individual boxes in the figure are be integrated together in any combination. Also as in the DNA-array example, the communication routes or topology (represented by the solid arrows) could be configured differently. As shown, a preferred embodiment is a user interface running on a client computer and that the client computer and the server (and the PCR/test unit, if there is a test portion) would all be hooked up to the Internet, and that the content or capture probe synthesizer is hooked into a LAN. However, any piece could be located locally or remotely and hooked up via LAN, Internet, etc.,—just as long as the various pieces can communicate appropriately, getting the information they need from other pieces.

Assume that the user wants to amplify up a set of DNA segments. Amplifying them in parallel saves steps over amplifying each piece one at a time. This scheme is implemented in the following steps. First, the user will specify which targets he or she is interested in PCR amplifying and possibly which are likely to be in the sample (the solution) he or she will be working with. Second, a server or servers (possibly with human intervention or help) will take the specification and design an array of PCR primers for the task. Third, the server will send the array specification to a primer-array synthesizer that will make the array. Fourth, after an array is made (perhaps that passes quality-control checks), the array is shipped to the user. Fifth, the user uses his or her sample or samples and the primers to do the requested amplification. Sixth, the PCR unit might be coupled to a unit for testing the results of PCR. For example, the results of PCR might be, by hand or by automation, put through gel electrophoresis and the results read, by a human or by automated machinery, to determine the quality of the PCR process. If the quality is unacceptable, the results can be integrated into a new design (either through a network directly or through interaction through the user interface) in step 2 above, and the rest of the steps can be repeated. Step 6 would not be done if the design process were not desired to be iterative at this level.

Some of the pieces can be somewhat different in some cases. For example, if the user specifies the primers for the array, there is no computational or design task to do in order to design the array. The server can simply transmit the data (perhaps with a simple reformatting) to the synthesizer system, or perhaps the user interface can transmit the data directly to the synthesizer system.

The data are transmitted over a network (such as the Internet, a company's internal LAN, etc.) or perhaps by transferring a disk or other removable media.

Step 1: Target Specification

The user would be interacting with target-specification software most preferentially through a Web browser interface or custom application (working over the Internet). This is shown in FIG. 2 as the “User Interface.”

The computational task can be easier (if the user is required to supply the full sequence of any material likely to be in solution and specifically which portions are to be amplified) or more complicated (if the user is allowed to specify sequences in a manner more open to some interpretation). For example, a user might specify a DNA sequence by an accession number from the GenBank database or by the full sequence as a text file. Or the user might specify something like “Human BRCA1 with the mutation 185delAG.” In this later case, the server side would need to do more processing to develop the DNA sequence of the target being specified, interacting with the a database to pull out the mRNA sequence for BRCA1, using the mutation specification to set the mutation, then using a database again to translate back into DNA.

Step 2: Array Design

Automated (or semi-automated) array-design software can implement mathematical models and heuristics to help speed the process of designing particular primers given a list of targets and possibly specific segments to amplify and what else might be in solution. The software designs a primer set or content that functions to selectively amplify targets. To do this, the designer (whether human or computer software) has to design each primer or primer pair sequence so that it hybridizes to its intended target sequence (and in the intended location, if that is also specified) but does not amplify (or at least not as well) unintended target sequences that might be in solution, including other primers.

Alternatively, array design might is an iterative process. For example, the user might specify targets or other initial input, view the result of the first pass at capture probe design and possibly some associated statistics or simulated hybridizations (or PCR amplifications) and results, and from that decide to change some input parameters, heuristics, or particular primers (to be designed again). This process might be repeated until the user is satisfied with the primer array. It might also be iterative on the level of the sixth step. Here a user would have gone through a previous design and previous PCR reaction and have tested or gotten some feedback on the results. These results then can be used to refine the design for another iteration of primers such as by indicating which primers from the previous run did not perform acceptably in amplifying their targets.

The design algorithm or process is represented in FIG. 2 as being internal to a server or servers at a (possibly) remote site; or, if there is user interaction at each design iteration, it is represented by the link from the server through the Internet back to the user's computer (which would be running a browser-type interface to a portion of the design software or perhaps custom front-end software that, again, would communicate to the server through the Internet) or by the link from the test of the PCR results. Or the server could be the user's own computer or a server at the user's site.

Step 3: Array Synthesis

After the array design is complete, the array specification is sent to a synthesizer (or synthesis factory or process) that then makes the capture probes.

Step 4: Ship to User

The array would most likely be checked for quality. Passed arrays could be sent via overnight courier to the user the next day.

Step 5: PCR Amplification

The user would put the array and the sample he or she is interested in into a PCR unit for the amplification process.

Step 6: View Results

### EXAMPLE 9

This example illustrates an experiment on an array be to take a sample solution containing genetic material and, for each SNP desired to be detected, either: (1) estimate that the sample solution is homozygous in the SNP, is homozygous in the wild type, or is heterozygous; or (2) classify the particular SNP as “uncallable” (i.e., cannot be classified according to (1) with confidence). One algorithm for designing an array to give such data is as follows. For each SNP sequence to be detected in the target solution, design three 17-mer probes where the first 17-mer is complementary to the wild type, the second 17-mer is complementary to the SNP, and the third 17-mer differs from both the first and second probes by one base, where all probes have the SNP location at their centers. For example, if the wild type and SNP sequences that would be in solution were respectively

| | |
|---|---|
| . . . ctgaataattactcaGctgaggtgagattt . . . | (wild |
| type) | |
| . . . ctgaataattactcaTctgaggtgagattt . . . | (SNP) |

(the capital letter shows the SNP location), one would construct the following three probes for the wild type, SNP, and control, respectively.

cacctcagCtgagtaat (wild type)
cacctcagAtgagtaat (SNP)
cacctcagTtgagtaat (control)

Assume that S is a measure of the strength that material in solution binds to a probe. In the case of an optical imaging system, S could be the optical intensity of a probe location after hybridization with a fluourescently labeled sample under stringent conditions (such that differences in binding based on single base-pair mismatches are measurable). Now one can map calls and the uncallable case to the following conditions.

If (0.80×S_wt)>S_snp and (0.80×S_wt)>S_control, call sample as homozygous wild type for that SNP.

If (0.80×S_snp)>S_wt and (0.80×S_snp)>S control, call sample as homozygous in the SNP.

If (0.80×S_wt<=S_snp<=S_wt/0.80) and (0.80×S_wt)>S_control and (0.80×S_snp)>S_control, call sample as heterozygous.

Otherwise, classify that particular SNP for that particular experiment as uncallable.

One could substitute in different values than 0.8 for the multipliers to get more or less restrictive calls.

In the case of deletions or insertions, put the location of the start of the insertion or deletion at the midpoint of an 18-mer, and change one of the bases immediately prior to the midpoint to make the control. This works for insertions or deletions of more than one base.

For example, if the wild type and SNP sequences that would be in solution were respectively

| | |
|---|---|
| . . . ctgaataattactcagctgaggtgagattt . . . | (wild |
| | type) |
| . . . ctgaataattactca-ctgaggtgagattt . . . | (SNP, |

deletion)

(the dash shows the location of the deletion), one would construct the following three probes for the wild type, SNP, and control, respectively.

tcacctcagCtgagtaat (wild type)
tcacctcagtgagtaatt (SNP)
tcacctcaTctgagtaat (control)

If the wild type and SNP sequences that would be in solution were respectively

| | |
|---|---|
| . . . ctgaataattactcag-ctgaggtgagattt . . . | (wild |
| | type) |
| . . . ctgaataattactcagActgaggtgagattt . . . | (SNP, |
| in | |
| sert) | |

(the dash shows the location of an insertion), one would construct the following three probes for the wild type, SNP, and control, respectively.

tcacctcagtgagtaatt (wild type)
tcacctcagTtgagtaat (SNP)
tcacctcaTtgagtaatt (control)

### EXAMPLE 10

One difficulty that can contribute to missed calls and increases in uncallable situations in example 9 has to do with the difficulty of developing conditions that are stringent for all probes at the same time. One way researchers have gotten around such issues is to use compounds such as TEAC or TMAC that mitigate the effect of A's and T's binding less strongly than G's and C's. These compounds produce a situation in which binding strength of two sequences depends more upon sequence length and less upon sequence itself. In this way, if one makes probes that are all the same length, stringency conditions will typically be more similar for all probes than if the compound were not used.

In the case where one does not use such balancing compounds (such as to reduce cost, to reduce toxicity of reagents, because hybridization might work better without it for a particular protocol that is already developed and tested in one's lab, etc.), another way that stringency can be balanced is to adjust the lengths of the probes so that their melting temperatures are similar. In this case, the algorithm for designing probes would be to start with the probes designed as in example 9 but then to increase or decrease lengths as necessary to get the wild-type probes to have the same estimated melting temperature within +/−2 C of a mean estimated melting temperature for 17-mers. Then the SNP and control probes would be set to have the same length and the same number of probes added to or subtracted from their ends as was done to the wild-type probe.

For example, let two wild-type/SNP sequence pairs that would be in solution be:

| | |
|---|---|
| . . . ctgaataattactcaGctgaggtgagattt . . . | (wild type 1) |
| . . . ctgaataattactcaTctgaggtgagattt . . . | (SNP 1) |
| . . . gggacgaccatatttatTtcaatcagatccatctg . . . | (wt 2) |
| . . . gggacgaccatatttatAtcaatcagatccatctg . . . | (SNP 2) |

Now construct the following trial set of probes.

cacctcagCtgagtaat (wild-type 1 probe)
cacctcagAtgagtaat (SNP 1 probe)
cacctcagTtgagtaat (control 1 probe)
ctgattgaAataaatat (wild-type 2 probe)
ctgattgaTataaatat (SNP 2 probe)
ctgattgaCataaatat (control 2 probe)

Using a nearest-neighbor melting-temperature model (such as the model discussed in Owczrzy et al. Biopolymers 44:217-239, 1997) with the parameters from Table III, column C, [Na+]=1 M, and strand concentration of 2 μM), the mean estimated melting temperature for 17-mers is approximately 69° C. The above wild-type probes have estimated melting temperatures under that model of 65.3° C. and 52.1° C., respectively. In this case, both probes need to be lengthened by adding bases alternately to each side (so that they remain complementary to the wild-type sequence in solution) until the estimated melting temperature is in the desired range.

For wild-type 1 probe, this process would yield:

| | | |
|---|---|---|
| aaatctcacctcagCtgagtaattattcag | | <-- complement of seq. |
| cacctcagCtgagtaat | 65.3° C. | <-- original probe |
| cacctcagCtgagtaatt | 66.3° C. | <-- one base added |
| tcacctcagCtgagtaatt | 68.2° C. | <-- two bases added |

So, the whole set of probes for detecting SNP 1 would become:

tcacctcagCtgagtaatt (wild-type 1 probe)
tcacctcagAtgagtaatt (SNP 1 probe)
tcacctcagTtgagtaatt (control 1 probe)

For wild-type 2 probe, this process would yield:

| | | |
|---|---|---|
| agatggatctgattgaAataaatatggtcgtccc | | <- - - complement of seq. |
| ctgattgaAataaatat | 52.1° C. | <- - - original probe |
| ctgattgaAataaatatg | 54.8° C. | <- - - one base added |
| tctgattgaAataaatatg | 57.1° C. | <- - - two bases added |
| tctgattgaAataaatatgg | 60.4° C. | |
| atctgattgaAataaatatgg | 61.3° C. | |
| atctgattgaAataaatatggt | 63.4° C. | |
| gatctgattgaAataaatatggt | 64.6° C. | |
| gatctgattgaAataaatatggtc | 65.7° C. | |
| ggatctgattgaAataaatatggtc | 68.0° C. | <- - - eight bases added |

So, the whole set of probes for detecting SNP 2 would become:

ggatctgattgaAataaatatggtc (wild-type 2 probe)
ggatctgattgaTataaatatggtc (SNP 2 probe)
ggatctgattgaCataaatatggtc (control 2 probe)

If the original trail wild-type probe had too high a melting temperature, bases would be alternately deleted off the ends of the probe until its estimated melting temperature were within the acceptable range. Then the SNP and control probes would have the same number of bases subtracted off their 5′ and 3′ ends as the wild-type probe did.

In this way, one can build up sets of probes that have approximately balanced estimated melting temperatures and thus are easier to manage under one set of conditions that will provide the needed stringency. Then one would do the same calling process as in example 9 (i.e., finding S_wt, S_snp, and S_control and applying the calling algorithm for each set of probes).

For cases of insertion and deletion, the process is the same except that, during extension, the bases added to the ends of a SNP probe to make it the same length as the new wild-type probe are such that the SNP probe remains complementary to the SNP sequence it is meant to capture (e.g., the bases added to the 3′ end might not be the same bases that get added to the 3′ end of the wild-type probe, although the number added would be the same).

### EXAMPLE 11

This example illustrates a probe design for gene expression assays. In the case of gene expression, what is typically desired is capture probes that selectively capture a particular gene's RNA (or cDNA) but do not capture as well other genes' RNA (or cDNA). In this way, if a probe captures something from solution, one assumes that what is captured is the particular target RNA (or cDNA) and not some other gene's RNA (or cDNA). In what follows, wherever the term “RNA” is used, the term “cDNA” could be substituted.

An example of an algorithm for design in this realm is as follows. The algorithm would be presented with a list of RNA sequences that it is to design probes for and a set of parameters as follows. The algorithm is to give M probes per target in the list. Each probe is to have an estimated melting temperature within a given range (Tmlow to Tmhigh) and to be a particular length (N). Also, each probe is to have a maximum melting temperature of simulated hybridization against any other gene's RNA in a database less than MCTmcrit. In this way, M probes are generated for each target (so that averaging of results can be used), and each probe is designed such that it has an estimated melting temperature against its intended target in the range of Tmlow to Tmhigh and a maximum estimated melting temperature against anything else in the database of MCTmcrit. If the estimated melting temperatures are accurate, one can heat the resulting array up to a temperature of MCTmcrit or higher but lower than Tmlow and cause miscaptures to denature while keeping hybridized correct bindings.

The algorithm accomplishes the selection of such probes as follows. For each target RNA, the following process is followed.

1. 1. Pick a location within the RNA at random.

2. 2. Increment the location by one base and consider this the start of an N-mer sequence. If our N-mer goes off the end of the RNA sequence, set the location to the first base in the RNA sequence. If we have already been at the first base in the RNA sequence, move on to the next RNA sequence—we can't find another probe for this sequence.

3. 3. Form the complement of the current N-mer segment. This is the candidate probe.

4. 4. The candidate probe's estimated Tm is calculated. If it falls outside the range Tmlow to Tmhigh, go to step 2.

5. 5. The candidate probe's MCTm is calculated (see below). If that value is greater than MCTmcrit, go to step 2.

6. 6. We now have an acceptable probe. Store it. If we have M probes stored, move on to the next RNA sequence. If not, go to step 2 and start on the next probe for this RNA sequence.

For the calculation of MCTm, do the following algorithm for each RNA sequence in the database other than the one the candidate probe was taken from as a complement.

1. 1. Start with the −(N−1)th base in the RNA sequence. (See below on positions)

2. 2. Set MCTm to −999.

3. 3. Align the candidate probe at the location picked in the RNA sequence.

4. 4. Calculate the estimated melting temperature of the candidate probe against that location of the RNA sequence.

5. 5. If the Tm value is greater than MCTm, set MCTm=the Tm value.

6. 6. If MCTm>MCTmcrit, exit the algorithm—this candidate probe will be thrown out, so there is no need to continue.

7. 7. Increment to the next base in the RNA sequence.

8. 8. If the location is past the end of the RNA sequence, exit—we are done.

9. 9. Go to step 3.

There are many models for calculating melting temperatures, including, for example, the model used in example 9 with the following modifications. First, the sequence used in the calculation is the maximum span of the probe that has associated bases in the target sequence. For example, with a probe of gattaca and a target sequence of tctgattgatataaatatggtc aligned at position 4 of the target sequence, we would have a binding arrangement of:

5′-tctgattgatataaatatggtc-3′
3′-acattag-5′

In this case, the whole probe sequence would be used for the Tm calculation. However, in the case of alignment at the −3^{rd} position, we would have:

5′-tctgattgatataaatatggtc-3′
3′-acattag-5′

In this case, only ttag would be used as the sequence to calculate Tm upon.

Second, as we are calculating based upon hybridization of one sequence against another sequence that is not necessarily exactly complementary, we need to use a Tm model that accounts for mismatches. For this, in the past we have used models such as those that reduce the calculated Tm by 1.5 C for every percentage of mismatch (e.g., if a 20-mer had 5 mismatches when compared to 20-mers worth of target that it was matched against, the estimate Tm would be the Tm that comes from the model of Example 1 minus 1.5×(5/20)×100, i.e., the calculated perfect-match Tm would be reduced by 37.5° C.). Or, in the case of

5′-tctgattgatataaatatggtc-3′
3′-acattag-5′

we would take ttag, calculate its melting temperature, and then reduce it by 1.5×(3/4)×100=112° C. as only 1 of its bases is complementary to the target. This is, of course, an extreme example of mismatches vs. sequence length for the Tm model. There are many other models for calculating Tm's taking into account mismatches, salt concentration, strand concentration, RNA/DNA vs. DNA/DNA binding, etc.

One inportant component of this whole process is the database against which one calculates the MCTm values. This database should at a minimum contain all of the RNA sequences in the original list, for which probes are desired. It is preferred that the database contains as many separate genes as possible, however, since in expression studies a sample might contain the expressions of many genes outside the list of what the researcher desires to study. One preferred candidate for this database, when working with human gene expression, is all of the cluster-representation sequences for the various clusters in Unigene. Also, between steps 4 and 5 of the main algorithm (the algorithm that starts with “1. Pick a location within the RNA at random”), one can add other constraints upon probes, picking other models and conditions to add into the process. For example, if one wants probes that are free of secondary structure, step 4b can be to calculate an estimate of secondary structure in the candidate probe and if it has an unacceptable amount, go to step 2.

We claim:

1. A process for a manufacturer to obtain customer orders for custom-designed biochips in an automated process, comprising obtaining desired target sequence(s) from the customer, wherein the target sequence(s) consist essentially of oligonucleotide sequences, polypeptide sequences, receptor binding site, or antigens to be bound; creating a sequence content motif for an array, wherein the sequence content motif consists essentially of oligonucleotide sequences, polypeptide sequences, or binding agents designed for complimentary binding; and applying the sequence content motif to a surface or within a porous matrix of a volume, suitable for later detection according to the sequence content motif, wherein the communication from the customer and the sequence content motif of each custom-designed biochip is retained within a storage device of the manufacturer.

2. The process for a manufacturer to obtain customer orders for custom-designed biochips of claim 1 wherein the desired target sequences are obtained from a database of sequences.

3. The process for a manufacturer to obtain customer orders for custom-designed biochips of claim 2 wherein the database of target sequences is selected from the group consisting of GenBank, TIGR, Incyte database, private databases and combinations thereof.

4. The process for a manufacturer to obtain customer orders for custom-designed biochips of claim 1 wherein the step of creating a sequence content motif comprises developing binding regions between a target sequence and a designed capture probe sequence according to consistent reaction conditions, wherein the reaction conditions include temperature and pH.

5. The process for a manufacturer to obtain customer orders for custom-designed biochips of claim 1 wherein the detecting step comprises exposing the custom-designed biochip to a sample to form an exposed custom-designed biochip, and either detecting binding with an instrumentation system designed to obtain a result at each site in a custom-designed biochip to obtain custom-designed biochip exposed data, or shipping the exposed custom-designed biochip back to the manufacturer to determine custom-designed biochip exposed data.

6. The process for a manufacturer to obtain customer orders for custom-designed biochips of claim 5 wherein the custom-designed biochip exposed data is analyzed by computer using a comparison to the sequence content motif for an array.

7. The process for a manufacturer to obtain customer orders for custom-designed biochips of claim 1 wherein the surface or the volume on which or within which a sequence content motif is applied is a selected from the group consisting of a solid non-porous surface, a silica-based surface, a porous matrix surface, a porous volume, a polysaccharide-based surface and layer, glass, and combinations thereof.

8. The process for a manufacturer to obtain customer orders for custom-designed biochips of claim 1 wherein the means for applying sequence content onto the surface or within the volume according to the content motif designed is selected from the group consisting of spotting oligonucleotides or polypeptides or in situ synthesis of oligonucleotides or polypeptides, photolithography of oligonucleotides or polypeptides or in situ synthesis of oligonucleotides or polypeptides, electrochemical-based pH changes in situ synthesis of oligonucleotides or polypeptides, photochemical-based pH changes for in situ synthesis of oligonucleotides or polypeptides, and combinations thereof.

9. The process for a manufacturer to obtain customer orders for custom-designed biochips of claim 1 wherein the surface on or volume in which a sequence content motif is applied is a selected from the group consisting of a solid non-porous surface, a silica-based surface, a porous matrix, a polysaccharide-based surface and layer, glass, and combinations thereof.

10. A system for a manufacturer to obtain customer orders for custom-designed biochips comprising a network-based receiving station for a manufacturer to receive desired target sequences from the customer, wherein the target sequences consist essentially of oligonucleotide sequence(s), polypeptide sequence(s), receptor binding site(s), or antigen(s) to be bound on a surface or within a porous matrix of a volume, or both; a software means for creating a sequence content motif for an array, wherein the sequence content motif consists essentially of oligonucleotide sequences, polypeptide sequences, or binding agents designed for complimentary binding; and a manufacturing system for applying the sequence content to a surface or within a volume or both, suitable for later detection according to the sequence content motif.

11. The system for a manufacturer to obtain customer orders for custom-designed biochips of claim 10 wherein the software means designs sequence content motif for binding to target of oligonucleotide sequence(s), polypeptide sequence(s), receptor binding site(s), or antigen(s) according to uniform melting temperatures, pH, environment, stringency conditions, or other conditions for consistent affinity binding of oligonucleotide sequence(s), polypeptide sequence(s), receptor binding site(s), or antigen(s).

12. The system for a manufacturer to obtain customer orders for custom-designed biochips of claim 10 wherein the system further comprises instrumentation for detecting binding of a sample onto the custom-designed biochip to generate exposure data, wherein the instrumentation resides at the customer or the manufacturer, at a third part or at multiple locations.

13. The system for a manufacturer to obtain customer orders for custom-designed biochips of claim 12 wherein the system further comprises the network or a new network for transmitting data showing binding on the custom-designed biochip to the manufacturer or designee for analysis of the sites according to the sequence content motif.

14. The system for a manufacturer to obtain customer orders for custom-designed biochips of claim 10 wherein the sequence content motif of each custom-designed biochip is retained within a storage device at the manufacturer.

15. The system for a manufacturer to obtain customer orders for custom-designed biochips of claim 10 wherein the desired target sequences are obtained from a database of sequences.

16. The system for a manufacturer to obtain customer orders for custom-designed biochips of claim 15 wherein the database of target sequences is selected from the group consisting of public databases, private databases, GenBank, TIGR, Incyte database, private databases and combinations thereof.

17. The system for a manufacturer to obtain customer orders for custom-designed biochips of claim 10 wherein the creation of content according to the sequence content motif comprises developing binding regions between a target sequence and a designed capture probe sequence according to consistent reaction conditions, wherein the reaction conditions include temperature, pH, stringency, ionic strength, hydrophilic or hydrophobic environment, and combinations thereof wherein a software program having melting temperature, stringency and proton (pH) chemistry algorithms is employed.

18. The system for a manufacturer to obtain customer orders for custom-designed biochips of claim 10 wherein the detecting step that exposes the custom-designed biochip to a sample to form an exposed custom-designed biochip, and either detecting binding with an instrumentation system designed to obtain a result at each site in a custom-designed biochip to obtain custom-designed biochip exposed data, or shipping the exposed custom-designed biochip back to the manufacturer to determine custom-designed biochip exposed data.

19. The system for a manufacturer to obtain customer orders for custom-designed biochips of claim 18 wherein the custom-designed biochip exposed data is analyzed by computer using a comparison to the sequence content motif for an array data as a template.

20. The system for a manufacturer to obtain customer orders for custom-designed biochips of claim 10 wherein the surface or volume having a porous matrix on which a sequence content motif is applied is a selected from the group consisting of a solid non-porous surface, a silica-based surface, a porous matrix, a polysaccharide-based surface and layer, glass, and combinations thereof.

21. The system for a manufacturer to obtain customer orders for custom-designed biochips of claim 10 wherein the means for applying sequence content onto a surface or within a porous matrix of a volume, or both, according to the motif designed, is selected from the group consisting of spotting oligonucleotides or polypeptides or in situ synthesis of oligonucleotides or polypeptides, photolithography of oligonucleotides or polypeptides or in situ synthesis of oligonucleotides or polypeptides, electrochemical-based pH changes in situ synthesis of oligonucleotides or polypeptides, photochemical-based pH changes for in situ synthesis of oligonucleotides or polypeptides, and combinations thereof.
